(19) United States
(12) Patent Application Publication
(10) Pub. No.: US 2005/0220838 A1
(43) Pub. Date: Oct. 6, 2005

(54) Medical device having surface depressions containing nitric oxide releasing compound

(76) Inventors: Yiju Zhao, Ellicot City, MD (US); William Herzog, Baltimore, MD (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/985,429

(22) Filed: Nov. 10, 2004

Related U.S. Application Data
(62) Division of application No. 10/291,753, filed on Nov. 12, 2002.
Application No. 10/291,753 is a continuation-in-part of application No. 09/254,002, filed on Mar. 1, 1999, now Pat. No. 6,656,217.
Application No. 09/254,002 is a continuation-in-part of application No. 08/703,646, filed on Aug. 27, 1996, now Pat. No. 5,797,887.

(51) Int. Cl. A61K 33/00; A61M 31/00

(52) U.S. Cl. 424/423; 424/608; 604/500

(57) ABSTRACT

A medical device including a surface, at least one depression in the surface, a nitric oxide releasing compound being deposited in the at least one depression, and at least one coating to cover the at least one depression. The coating forms a barrier inhibiting release of the nitric oxide releasing compound and being permeable to nitric oxide when the device is inserted in bodily fluid.

### RELATED APPLICATIONS

This application is a divisional application claiming priority to currently pending U.S. application with the Ser. No. 10/291,753, which was filed Nov. 12, 2002, which is a continuation-in-part of U.S. Pat. No. 6,656,217, which is a continuation-in-part of issued U.S. Pat. No. 5,797,887.

This application is a continuation-in-part of U.S. application Ser. No. 09/254,002 filed Mar. 1, 1999, which is a national stage application of PCT/US97/15022 filed Aug. 27, 1997, which claims priority of U.S. application Ser. No. 08/703,646, filed Aug. 27, 1996 and issued as U.S. Pat. No. 5,797,887. Each of the U.S. application Ser. Nos. 09/254,002 and 08/703,646 and PCT/US97/15022 is hereby incorporated in its entirety by reference.

### BACKGROUND OF THE INVENTION

This invention relates generally to novel drug delivery devices containing a nitric oxide releasing compound entrapped in surface modifications of the devices and methods for using them.

Nitric oxide releasing compounds such as sodium nitroprusside (SNP) and similar nitrosyl-containing organometallic compounds, whether ionic salts or chelates, which can release nitric oxide (NO) upon light activation and/or temperature activation, have been known to relax vascular smooth muscle tone and may exhibit short-term hypotensive effects. Besides regulating vascular tone, nitric oxide has been found to control a wide variety of physiological functions, including (a) inhibition of neutrophil adhesion, (b) enhancement of macrophage-mediated microbial killing, (c) amelioration of impotence, (d) regulation of various CNS functions, (e) inhibition of platelet adhesion/aggregation, and (f) inhibition of smooth muscle cell proliferation (and thereby inhibit restenosis after angioplasty).

Pharmacological applications of nitric oxide released from nitric oxide releasing compounds are limited. Sodium nitroprusside, for example, is used therapeutically for the short term (24-72 hours) treatment of hypertensive emergencies. The degradation of sodium nitroprusside is attributed to reductive processes taking place in the bloodstream. Even though it has been suggested that sulfhydryl groups attached to endothelial cells lining the vascular walls might initiate this reaction, other reductants such as glutathione or ascorbic acid may likewise contribute to its unusually short physiological lifetime. Based on this pharmacological behavior, typical use of this drug requires it to be given continuously as an intravenous solution, or it rapidly loses its efficacy resulting in a return of blood pressure to a hypertensive level. This characteristic makes sodium nitroprusside relatively difficult to monitor and control in the therapeutic setting. Because this nitric oxide releasing compound has a short lifetime of several minutes in blood, its use is limited to acute hospital-based intensive care unit treatment of hypertensive emergencies.

Systemic administration of gaseous nitric oxide to treat localized abnormalities or diseases is likewise limited by delivery systems which are difficult to control and thus require close monitoring. For example, inhaled gaseous nitric oxide is used on rare occasions to treat pulmonary hypertension. This is typically only performed in an intensive hospital care setting because control of its dosage in the therapeutic range to avoid systemic toxicity is hard to achieve. Even when possible to carefully titrate the gaseous dose of nitric oxide to minimize systemic toxicity, it is very difficult to locally administer the drug to particular sites.

Several apparatuses and methods have been developed for delivering drugs selectively and locally to a specific internal body site.

For instance, U.S. Pat. No. 5,282,785 employs a drug delivery apparatus comprising a flexible catheter for insertion into an internal target area of the body and a drug delivery means connected to the catheter.

U.S. Pat. No. 5,286,254, also employs an apparatus comprising a drug delivery means having a fluid delivery passageway for delivering a drug to the distal end of the apparatus.

These types of apparatuses described in U.S. Pat. Nos. 5,282,785 and 5,286,254 have several disadvantages. These catheter-based devices obstruct blood flow and therefore cannot stay in the circulation system very long. Therefore, long-term drug delivery is not possible using these systems. The presence of these items in the circulatory system promotes platelet deposition on the device.

U.S. Pat. No. 5,605,696 teaches that a polymer into which a therapeutic drug is incorporated therein is coated onto a stent. A rate-controlling membrane can also be applied over the drug loaded polymer to limit the release rate of the therapeutic drug.

### SUMMARY OF THE INVENTION

The present invention relates to a medical device including a surface, at least one depression in the surface, a nitric oxide releasing compound being deposited in the at least one depression, and at least one coating to cover the at least one depression. The coating forms a barrier inhibiting release of the nitrosyl-containing organometallic compound and being permeable to nitric oxide when the device is inserted in bodily fluid.

### BRIEF DESCRIPTION OF THE INVENTION

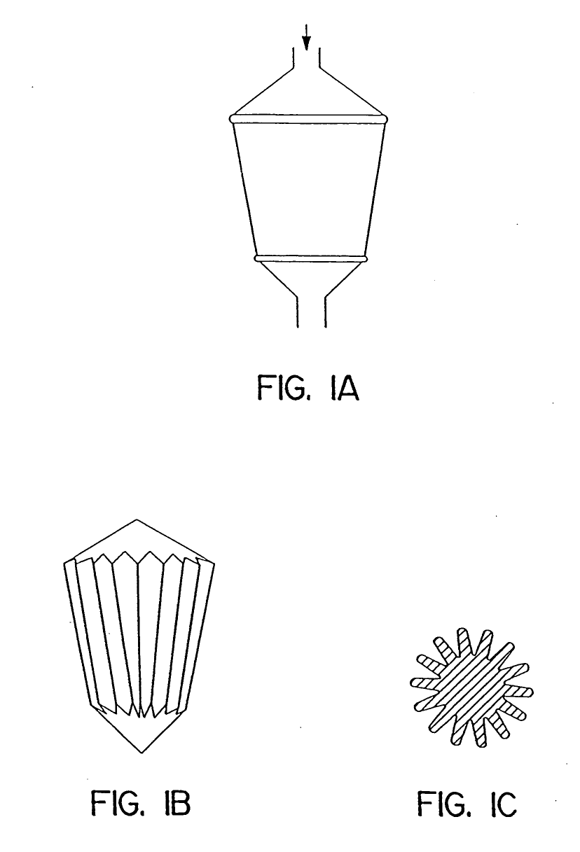

FIGS. 1A-C illustrate the exterior, interior and cross sectional views, respectively, of an exemplary platelet-inhibition element according to an embodiment of the present invention, which comprises a container adapted to be inserted in the blood flow loop of a patient.

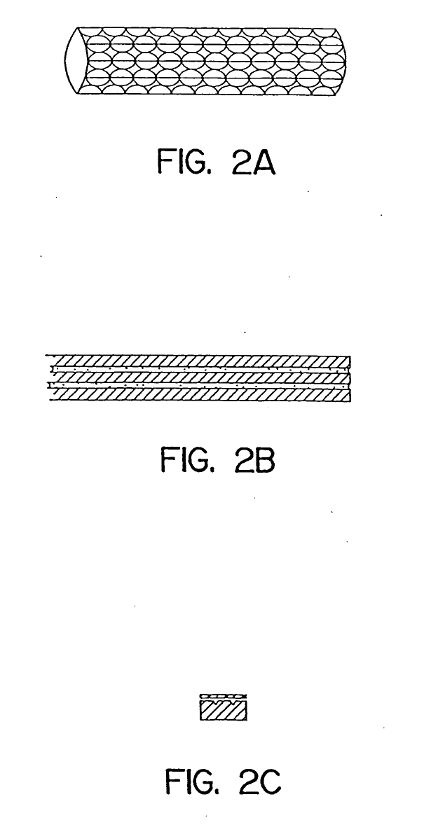

FIG. 2A illustrates the side view of a stent according to an embodiment of the present invention.

FIG. 2B illustrates a strand of the stent in FIG. 2A.

FIG. 2C illustrates the cross-sectional view of the strand of the stent in FIG. 2B.

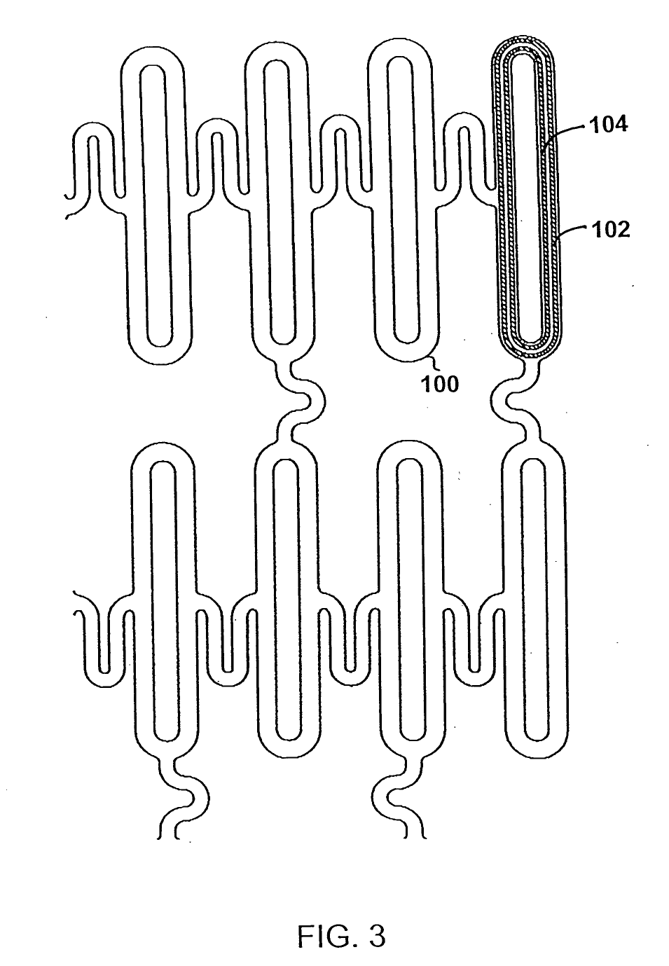

FIG. 3 illustrates another exemplary embodiment of a stent according to the present invention with channels formed on the stent.

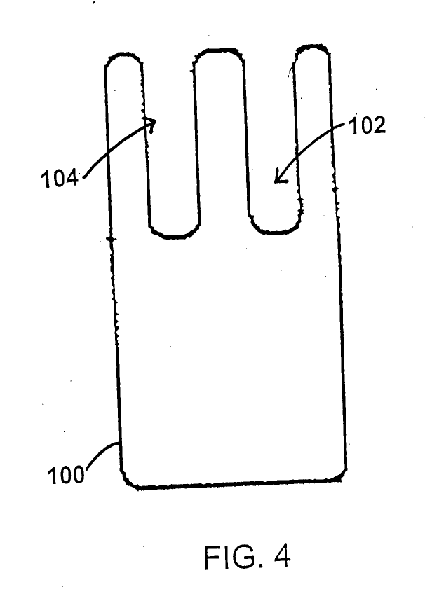

FIG. 4 illustrates an exemplary cross-sectional view of the stent in FIG. 3.

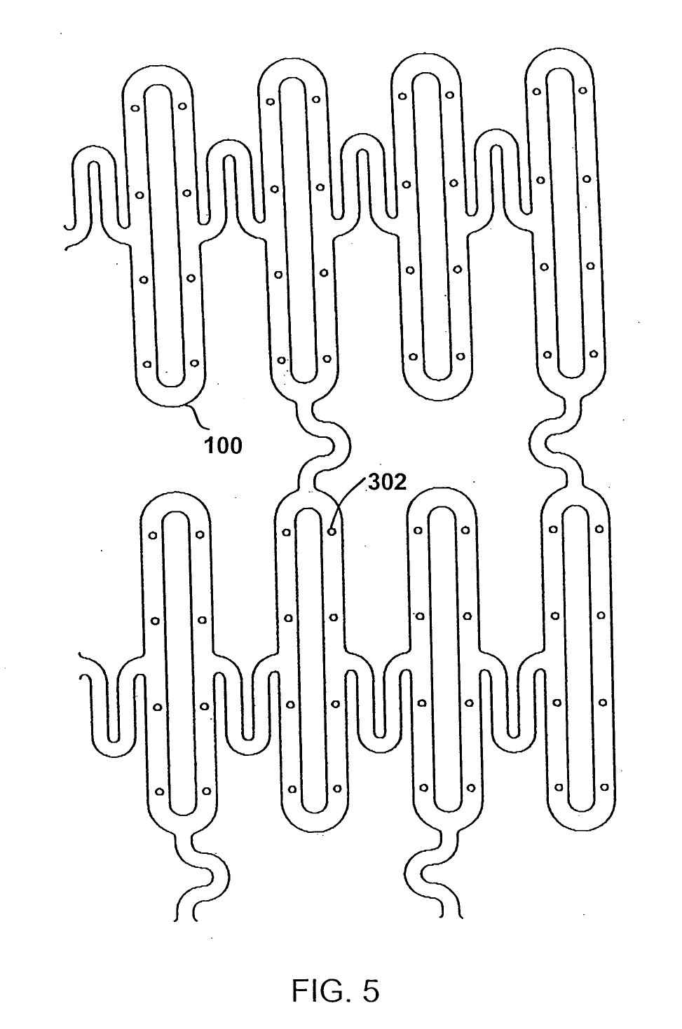

FIG. 5 illustrates another exemplary embodiment of a stent according to the present invention with perforations formed on the stent.

### DETAILED DESCRIPTION

FIGS. 1A-C illustrate the exterior, interior and cross sectional views, respectively, of an exemplary medical device for platelet-inhibition according to an embodiment of the present invention. The device comprises a container in FIG. 1A adapted to be inserted in the blood flow loop of a patient undergoing renal dialysis or surgery involving extravascular transport of the blood stream of the patient. An accordion folded biologically inert synthetic polymer mesh insert in FIG. 1B is placed inside the container through which the blood of the patient flows. The outer surface of the synthetic polymer mesh and the inner suface of the container may be depressed, i.e., grooved or perforated, for depositing nitro oxide releasing compounds.

FIG. 2A illustrates a side view of an exemplary medical stent according to an embodiment of a medical device of the present invention. FIG. 2B illustrates a strand of the medical stent in FIG. 2A and discloses depressions in the inner walls thereof for deposition of nitric oxide releasing compounds. FIG. 2C illustrates the cross-sectional view of the strand of the stent in FIG. 2B, where two V-shaped channels for depositing nitric oxide releasing compounds and a layer of coating on top of the channels are disclosed.

A medical device according to the present invention may have a coating on its surface to which circulating blood is exposed and which covers or is impregnated with (i.e., dispersed with or dissolved with) a nitric oxide releasing compound. The nitric oxide releasing compound, whether an ionic salt or a chelate, is stable at room temperature but at body temperature and/or in the presence of ambient light while the medical device is exposed to the blood with blood-born reductances, releases a platelet-aggregation-inhibiting amount of nitric oxide. Such released nitric oxide penetrates via the coating and produces a nitric oxide concentration locally at the surface of the medical device.

A medical device according to the present invention may be any intravascular or extravascular device, that contacts blood. Intravascular medical devices may include synthetic (prosthetic) grafts (vascular or non-vascular), implantable pumps, heart valves and stents adapted for long term or permanent insertion into the lumen of a blood vessel, e.g., in conjunction with percutaneous transluminal angioplasty. The intravascular devices may include ones adapted for temporary insertion in a blood vessel, e.g., a balloon or catheter tip.

Extravascular medical devices may include a lumen (interior wall) of a plastic tubing or a membrane insert in an extravascular path of the blood stream of a living being undergoing a medical procedure that requires the cycling of the blood stream or a portion thereof outside the body of the living being, e.g., a coronary artery bypass surgery (cardiopulmonary bypass) or renal, kidney dialysis.

In a medical device according to the present invention, whether intravascular or extravascular, an applicable surface of the device has a coating as described herein which covers or is impregnated with at least one nitric oxide releasing compound as described herein.

The coating may include any feasible coating such as polymeric coating having pores with a porosity sufficiently low to inhibit the diffusion of the nitric oxide releasing compound from or through the coating into the blood stream and also to inhibit blood-borne reductants from entering the coating. The coating is gas permeable and does not prevent the diffusion of nitric oxide produced from the nitric oxide releasing compound into the blood stream. The coating may be permeable to nitric oxide only or may also be permeable to other gases.

By exposing such coated surface to the blood stream of a living being, nitric oxide is released from the coating in a controlled manner while retaining the other non-volatile decomposition products within the polymer coating.

The coating on a medical device according to the present invention may be about 0.1-1.0 mm thick and may contain about 1-100 micromoles of a nitric oxide releasing compound per mm². Even higher concentrations can be used when the diffusion rate of the nitric oxide or longer release of the nitric oxide are desired.

Other exemplary polymers according to the present invention includes physiologically inert and biodegradable polymers, synthetic polymers, and those which are only slowly soluble or insoluble in blood while any portion of the nitric oxide releasing compound remains covered by or impregnated within the coating. Exemplary insoluble polymers according to the present invention are those which form a gas-permeable membrane coating around the medical device. Examples of biodegradable polymers according to the present invention include natural polymers such as collagen, albumin, casein, fibrin and gelatin. Synthetic polymers according to the present invention include polylactide, polyglycoside, polyvinyl alcohols, polyalkylene oxides and polyvinyl chlorides. Other suitable polymers according to the present invention include polyesters, polylactic anhydrides, celluloses, vinyl copolymers, homopolymers, acrylate, polycyanoacrylate, polyurethanes, silicone polymers and other types of polymers, such as dendrimers.

The coating according to the present invention may have one or more of the following characteristics: being applicable to luminal or subluminal surfaces; not causing a significant increase in stent wall thickness; being stable over time without desquamation; having a surface tension below 30 dyne/cm; having a smooth surface texture (<1 micron irregularities); having a negative or neutral surface charge; allowing rapid endothelialization; permitting timed elution of nitric oxide; and delivering an effective concentration of nitric oxide locally to the site.

Applicable surfaces of a medical device according to the present invention may be covered by a coating of the present invention by immersing the surface in a solution or dispersion of a selected polymer in either an aqueous or an organic vehicle which may or may not be impregnated with a nitric oxide releasing compound, and then making the coating insoluble, e.g., by changing the pH or the ionic strength, by evaporation of the solvent or by denaturing a proteinaceous polymer, so that a coating of the polymer deposits on the exposed surfaces of the medical device. For example, a stent according to the present invention may be placed in a tetrahydrofuran (THF) solution of polyvinyl chloride (PVC) which may or may not be impregnated with a nitric oxide releasing compound. The surface of the stent is thereby coated with a solution of THF/PVC which may or may not be impregnated with a nitric oxide releasing compound. Upon evaporation of the solution, the polymer forms a film on the surface of the stent over the depressions.

Surface depressions according to the present invention may be formed as part of a surface of a medical device or may be formed on the surface after the device is formed. According to the present invention, surface depressions can be filled with a polymer containing a nitric oxide releasing compound according to the present invention. Alternatively, a nitric oxide releasing compound without such polymer may be deposited in surface depressions and coated with a polymer which may or may not contain the organometallic compound. A second coating can be applied on top of the first coating, where the second coating may be formed from the same polymer or a different polymer and may or may not be impregnated with a nitric oxide releasing compound.

Nitric oxide releasing compounds, whether ionic salts or chelates, according to the present invention may be non-toxic, that is, substantially free from any significant toxic effects at their effective applied concentration. The nitric oxide releasing compounds according to the present invention may also be substantially free of symptomology, i.e., they do not produce significant symptoms detectable to the person treated at their effective applied concentration. Further, the nitric oxide releasing compounds may be relatively stable at room temperature, away from heat and light, i.e., once a nitric oxide releasing compound is covered by or is impregnated into, for example, a polymer coating, nitric oxide is not released therefrom at a significant rate. During the application of nitric oxide releasing compound to depressions and/or a coating of a medical device according to the present invention, or thereafter, during self storage in a packaged container, nitric oxide is released at a rate, for example, less than 1% per month. The duration of the delivery of nitric oxide, when the medical device according to the present invention is placed in contact with bodily fluid such as blood, can be adjusted by varying the concentration or amount of the nitric oxide releasing compound covered by or impregnated in the coating. The delivery of nitric oxide can last a matter of minutes, (e.g., 5-90 minutes in the case of a angioplasty balloon or catheter), hours (e.g., 1-4 hours in the case of hypothermic surgery blood circulation or cardiopulmonary bypass), days (e.g., 3 hours to 3 days in the case of dialysis of blood passing though plastic tubing), or weeks (e.g., 4 to 6 weeks or longer in the case of a stent). Different types of nitric oxide releasing compounds may be deposited in a surface depression or a plurality of surface depressions according to the present invention in order to achieve different nitric oxide releasing properties.

Nitric oxide can be locally delivered at any desired dose profile, which can be controlled primarily by varying the volume of surface depressions, the concentration or amount of the nitric oxide releasing compound, the specific polymer used to form or the nature and thickness of the coating, e.g., by employing multiple polymer coatings containing varying concentrations of a nitric oxide releasing compound.

The examples of a nitric oxide releasing compound employed in this invention may include a compound of the formula [MX₅NO]⁻²Y⁻² or 2Y⁺¹ where M is a transition metal such as Fe, Co, Mn, Cu, Ni, Pt, X is a negatively charged ion such as CN, Cl, Br, I, or chelates such as EDTA, DTPA, carbamates and dithiolates that at physiological pH have negatively charged carboxylic and thiocarboxylic acid groups, and Y is a positively charged salt.

A readily available example of a nitric oxide releasing compound that can be employed in the present invention may be different types of nitrosyl-containing organometallic compounds such as sodium nitroprusside, which is a compound in which an iron ion is complexed to five cyano groups and the sixth ligand position is occupied by a nitrosyl group.

Other suitable complexing agents for the iron ion are ethylenediaminetetraacetic acid (EDTA); diethylenetriaminepentaacetic acid (DTPA) and others of this class of chelates; 1,4,7,10-tetraazacyclododecane-N,N,N′,N″,N′″-tetraacetic acid, DOTA and trans-1,2-cyclohexylenediamine-N,N′,N′-tetraacetic acid and others of this class of chelates; diethylthiocarbamate and similarly related carbamates; 1,2-dicyanoethylene-1,2-dithiolate and similarly related dithiolates.

The medical device may be synthetic or reconstituted natural, e.g., from powdered bone and binder, which can trigger a foreign body response and therefore can benefit from surface depressions according to this invention. The foreign body can also be a metal (e.g., stainless steel).

The present invention may inhibit platelet aggregation, either in the form of a layer that builds up on a medical device that is permanently implanted in a blood vessel or that comes in contact with the circulating blood of a living being on a temporary basis or in the form of a detachable clot which, if it travels to the organs such as brain, lung, heart, kidney and liver, can be debilitating or have life-threatening consequence.

The present invention may also inhibit restenosis, i.e., a gradual re-occlusion of the blood vessel over a prolonged time period after surgery, frequently 4 to 6 weeks, by coating the surface of the foreign body such as a stent that contacts the blood with a polymer coating disclosed herein which covers or is impregnated with a nitric oxide releasing compound.

Surface depressions according to the present invention can be in any form including channels, grooves, holes and perforations. The depressions can be machined, cut, etched or otherwise placed on a wall of the medical device. The depressions can be created during or after the original manufacturing of the medical device. The depressions may be formed locally or pervasively over at least one applicable surface of a wall of the medical device. The depressions may extend partially or completely through a wall of the medical device. The depressions may be of any geometric shape and size. The depressions may all be of a uniform size and/or shape or may vary in size and/or shape. The depressions may be arranged in any pattern.

In FIGS. 3 and 4, channels 102 and 104 formed on the stent 100 may have a cross-section of any geometric shape including a U-shape, V-shape, rectangular shape or a semicircular shape. The channels 102 and 104 may be cut/etched as parallel, perpendicular or skewed series to the stent's design or in any other patterns. The channels may be cut/etched locally or pervasively over at least one applicable surface of a strand of the stent. The stent may have only one, two or larger number of channels formed thereon. The channels may extend partially or completely through a strand. The exemplary embodiment of the channel modification in FIGS. 3 and 4 shows a series of two “U,” shaped channels parallel to the stent's design. Etching technology and various cutting technologies including electro-discharge machining and laser-cutting may be used to form the channels. The channels may extend about 40%, ⅓, ¼, ½ or any other proportion of the depth of a stent strand. In FIG. 4, which shows a cross section of three ears formed by the two channels 102 and 104 of a strand of the stent 100, the strand may be about 0.09 mm in width and may be about 0.1651 mm in height. Each of the two channels 102 and 104 may be about 0.02 mm in width. The two side ears may be about 0.015 mm in width, the center ear may be about 0.02 mm in width, and the ears may be about 0.07 mm in height.

FIG. 5 shows a stent 100 having perforations 302. The perforations 302 may be of circular, rectangular, square, oval, star, triangular, or any other geometry and any size. The perforations 302 may be uniform in size and/or shape or may vary in size and/or shape. They may extend completely through the depth of a wall of the stent to create holes in the stent or extend partially. A strand may be about 0.07 mm in width. Each perforation 302 may be about 0.015 mm in radius and may be distanced from a neighboring perforation by about 0.4 mm. Each of the depicted loops formed in the stent 100 includes two straight portions connected at their ends by two semicircular portions as shown in FIG. 4. Each loop may be about 0.2937 mm in diameter across the two straight portions and may be about 1.6 mm in diameter across the semi-circular end portions. The perforations 302 may be formed in any pattern and may be formed locally or pervasively over one or more applicable surfaces of the stent 100. In FIG. 4, surface perforations may be a series of circular perforations, where a diameter of at least one of the perforations may be about ⅓, ¼, ½ or any other proportion of the width of the stent. The perforations may be centered or set to one side on the width of the strand and evenly or unevenly placed along its length. The perforations 302 may be made to the stent 100 by any method including electro-discharge machining, laser-cutting, any other cutting technology, and etching technology.

After the stent 100 in FIGS. 3-5 is modified in the described manner it may be covered with a coating according to the present invention. The coating may be applied locally or pervasively over at least one applicable surface of the stent 100. Nitric oxide releasing compounds may be placed inside the channels or perforations. In that case, the coating will cover the nitric oxide releasing compounds.

From the foregoing description, one skilled in the art can easily ascertain the essential characteristics of this invention, and without departing from the spirit and scope thereof, can make various changes and modifications of the invention to adapt it to various usages and conditions. The entire disclosures of all patents cited above are incorporated by reference. The following preferred specific embodiments are, therefore, to be construed as merely illustrative and not limiting to the disclosure in anyway whatsoever.

1-34. (canceled)

35. An NO releasing system comprising a substrate upon which is disposed a matrix having a dissolved NO donor, which matrix releases NO at a maximum daily rate on a given day, and releases at least 10% of the maximum daily rate one week after the given day.

36. The system of claim 35 wherein the matrix releases at least 10% of the maximum daily rate two weeks after the given day.

37. The system of claim 35 wherein the substrate, matrix and NO donor is disposed in an physiologic environment, and the concentration of released NO drops by no more than one order of magnitude over a two week period.

38. The system of claim 35 wherein releasing of the NO is measured in a flow system test assay using 5 ml of phosphate buffer solution, re-circulating at 100 ml/min, at room temperature.

39. The system of claim 35 wherein the matrix is substantially not bio-absorbable.

40. The system of claim 35 wherein the matrix is solid and substantially hydrophobic.

41. The system of claim 35 wherein the NO donor comprises a nitroprusside.

42. The system of claim 35 wherein the daily release rate is substantially independent of hydrolysis of the NO donor in the matrix.

43. The system of claim 35 wherein the system releases a total of at least 10 nmoles of NO.

44. The system of claim 35 further comprising a coating over the matrix that has a different chemical composition from the matrix, and that assists in attenuating transport of a reductant into the matrix.
